(19) United States
(12) Patent Application Publication
(10) Pub. No.: US 2010/0151416 A1
(43) Pub. Date: Jun. 17, 2010

(54) INTERACTIVE PATIENT IMMOBILIZATION SYSTEM

(75) Inventors: Siyong KIM, Gainesville, FL (US); Richard D. Helmig, Gainesville, FL (US)

Correspondence Address: MILES & STOCKBRIDGE PC, 1751 PINNACLE DRIVE, SUITE 500, MCLEAN, VA, 22102-3833, US

(73) Assignee: University Of Florida

(21) Appl. No.: 12/715,021

(22) Filed: Mar. 1, 2010

Related U.S. Application Data
(63) Continuation of application No. 10/464,866, filed on Jun. 19, 2003.
(60) Provisional application No. 60/389,719, filed on Jun. 19, 2002.

(51) Int. Cl. A61C 3/00; A61B 18/20

(52) U.S. Cl. 433/140; 606/13

(57) ABSTRACT

A device for positioning a patient in a fixed location comprising:
a) a locator attachable to a patient and having a registration portion for registration with a portion of a patient's body, and
b) means for transmitting information concerning the position of the portion of the patient to an information receiving means, the means being rigidly connected with the locator; a system for positioning a patient in a fixed location comprising the above device in combination with means for receiving said information concerning the position of said portion of said patient; and

a method for locating a portion of a patient in a predetermined fixed position comprising:
a) affixing to a patient the above device by registering the registration portion of the device with the portion of the patient,
b) transmitting information concerning the position of the portion of the patient from the device to an information receiving means, and
c) locating the portion of the patient in the predetermined fixed position based on the transmitted information.

### BACKGROUND OF THE INVENTION

1. Field of the Invention

The invention relates to methods and systems for positioning patients for surgical and other medical procedures.

2. Description of the Prior Art

Conventional radiation treatment typically involves directing a radiation beam at a tumor in a patient to deliver a predetermined dose of therapeutic radiation to the tumor according to an established treatment plan. A suitable radiation treatment device is described in U.S. Pat. No. 5,668,847, issued Sep. 16, 1997 to Hernandez, the contents of which are incorporated herein for all purposes.

Healthy tissue and organs are often in the treatment path of the radiation beam during radiation treatment. The healthy tissue and organs must be taken into account when delivering a dose of radiation to the tumor, thereby complicating determination of the treatment plan. Specifically, the plan must strike a balance between the need to minimize damage to healthy tissue and organs and the need to ensure that the tumor receives an adequately high dose of radiation. In this regard, cure rates for many tumors are a sensitive function of the radiation dose they receive.

Treatment plans are therefore designed to maximize radiation delivered to a target while minimizing radiation delivered to healthy tissue. However, a treatment plan is designed assuming that relevant portions of a patient will be in a particular position during treatment. If the relevant portions are not positioned exactly as required by the treatment plan, the goals of maximizing target radiation and minimizing healthy tissue radiation may not be achieved. More specifically, errors in positioning the patient can cause the delivery of low radiation doses to tumors and high radiation doses to sensitive healthy tissue. The potential for misdelivery increases with increased positioning errors.

Due to the foregoing, treatment plans are designed under the assumption that positioning errors may occur that may result in misdelivery of radiation. Treatment plans compensate for this potential misdelivery by specifying lower doses or smaller beam shapes (e.g., beams that do not radiate edge of a tumor) than would be specified if misdelivery was not expected. Such compensation may decrease as margins of error in patient positioning decrease.

Current radiation treatment devices provide sophisticated control over radiation delivery to a patient site. Specifically, these devices allow a therapist to target a tumor with Intensity-Modulated RadioTherapy (IMRT) treatments, Conformal Radiation Treatments (CRT) and composite radiation beam distributions. However, as described above, the full effectiveness of such features cannot be achieved without a system providing accurate patient positioning.

When used in conjunction with conventionally-designed treatments, more accurate positioning reduces the chance of harming healthy tissue. More accurate patient positioning also allows the use of more aggressive treatments. Specifically, if a margin of error in patient positioning is known to be small, treatment may be designed to safely radiate a greater portion of a tumor with higher doses than in scenarios where the margin of error is larger.

Modern radiation treatments provide the delivery of multiple radiation beams during the course of treatment. A treatment is divided into multiple fractions, with each fraction being delivered to a patient according to a periodic schedule such as weekly or the like. Each fraction consists of multiple segments, with each segment specifying a particular beam type, beam shape, dose, treatment device position, and delivery time. Of course, two segments of a fraction need not differ in each of the above factors.

During a treatment fraction, adjustments must be made after each segment to the treatment device and/or to the patient position. These adjustments are often time-consuming, because most radiation therapy devices are located within vaults constructed with thick concrete walls and thick doors that can take 30 seconds to open and close. Therefore, it can take a significant amount of time after a segment is completed for an operator to enter the room, make the necessary adjustments, leave the room, and operate the radiation treatment device to deliver the next segment.

Intensity modulation radiation therapy (IMRT) enables the treatment of lesions that either partially or fully surround critical normal tissues. It requires a high degree of precision, both in set-up and positioning of the patient to achieve the full benefit of IMRT. However, even though the patient can be set up accurately initially, the probability of patient movement during the entire treatment of fraction (intra-fraction movement) is much higher in IMRT compared to conventional therapy because the total treatment time of IMRT is enormously longer.

Patient movement can be reduced with the aid of immobilization or positioning devices. Currently, the thermoplastic mask is the most common method for immobilization during brain, head and neck treatments. The mask system has been shown to restrict patient movement efficiently and accurately for conventional treatments. However, there are a few issues to consider in the use of mask system only for high precision therapy like IMRT treatments. First, mask is somewhat uncomfortable for many patients. Comfort is a very important factor in IMRT because of the much longer treatment times involved. Although an uncomfortable immobilization device may work properly in relatively short treatments, it may cause problems in cases where patients strain to move even more after a certain period of time due to lack of comfort when treatment times are very long. Some patients who experience claustrophobia cannot tolerate the mask. Another problem is that many head and neck patients lose weight significantly during the treatment period, resulting in loose masks. The adverse effects of using loose-fitting masks is severe in IMRT. It is often necessary to construct a new mask periodically as the original mask becomes too loose fitting, which may require re-scan, re-plan, and QA for IMRT, thereby increasing costs. The Vac Fix® mold-strap combination system is also used for the immobilization or positioning of a patient. In this system, the patient's head comfortably fits to a customized Vac Fix® mold and is fastened by straps. This system cooperates with a patient movement monitoring system. [Bova et al, The University of Florida frameless high-precision stereotactic radiotherapy system. Int J Radiat Oncol Biol Phys 1997; 38(4):875-882; Buatti et al, Preliminary experience with frameless stereotactic radiotherapy, Int J Radiat Oncol Biol Phys 1998; 42(3):591-592; Meeks et al. Image localization for frameless stereotactic radiotherapy, Int J Radiat Oncol Biol Phys 2000; 46(5):1291-1299, and Tome et al. A high-precision system for conformal intracranial radiotherapy, Int J Radiat Oncol Biol Phys 2000; 47(45): 1137-1143.]

However, all immobilization systems currently available are passive from the patient's point of view. Patients are asked to remain immobile once the initial setups are done. But by nature, patients are always restless and prone to move, resulting in a certain amount of displacement of the target throughout the procedure. It is then necessary to re-setup the patient if the displacement is out of the tolerance incorporated in the plan of treatment. Unfortunately, however, it is very difficult to detect the amount of patient displacement that occurred during the fraction treatment in the mask system. Real time monitoring systems such as cameras, for example, provide sufficient information to perform patient re-setup with relative ease. However, such procedures are very time consuming and labor intensive since the beam needs to be discontinued and the therapists must re-enter the treatment room and re-setup the patient whenever the displacement is out of tolerance. Moreover, the necessity for these repeated procedures increases the time the patient is required to remain immobile, thereby increasing the likelihood of further displacements of position due to restlessness.

It is an object of the invention to provide a system and method of patient positioning that is not subject to the above-noted disadvantages.

### SUMMARY OF THE INVENTION

One embodiment of the invention relates to a device for positioning a patient in a fixed location comprising:
a) a locator attachable to a patient and having a registration portion for registration with a portion of a patient's body, and
b) means for transmitting information concerning the position of the portion of the patient to an information receiving means, the means being rigidly connected with the locator.

A second embodiment of the invention concerns a system for positioning a patient in a fixed location comprising the above device in combination with means for receiving the information concerning the position of the portion of the patient.

A still further embodiment of the invention relates to a method for locating a portion of a patient in a predetermined fixed position comprising:
a) affixing to the patient the above device by registering the registration portion of the device with the portion of the patient,
b) transmitting information concerning the position of the portion of the patient from the device to an information receiving means, and
c) locating the portion of the patient in the predetermined fixed position based on the transmitted information.

### BRIEF DESCRIPTION OF THE DRAWINGS

FIG. 1 depicts the patient positioning device of the invention.

FIGS. 2-4 depict schematic representations of various embodiments of the invention in use.

### DETAILED DESCRIPTION OF THE INVENTION

One embodiment of the present invention relates to an interactive positioning system and device, a new concept in patient immobilization in radiation therapies particularly adapted for brain, neck and head treatments. The term, “interactive positioning process”, as used herein, defines a process that provides information of patient movement during treatment to patients in real time and allows them to feedback their own motion to maintain the desired setup position as closely as possible. One embodiment of the system comprises the above described device having means thereon to transmit a tracing laser and a target laser.

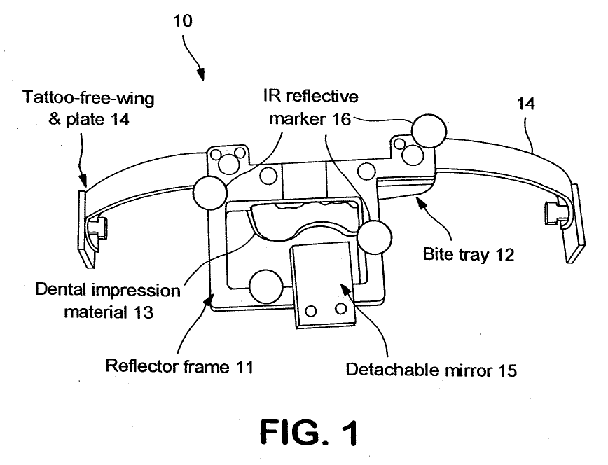

The device of the invention is illustrated in FIG. 1. The device 10 comprises frame 11 having rigidly attached thereto a bite tray or plate 12. Affixed to the bite tray 12 is a dental impression material 13. In use the patient grasps the impression material in his teeth thereby imprinting the material with a bite impression that is permanent upon hardening of the impression. Thereafter, each time the patient grasps the device by registering his/her bite with the impressed pattern on the bite tray, the device is always oriented on the patient in the same position. The frame is also provided with tattoo-free wings 14. A detachable laser-reflecting mirror 15 is also rigidly attached to the frame 13. Optionally, IR-reflective markers 16 may also be affixed to the frame 11 and/or the wings 14. Alternatively, a laser generator (not shown) may be substituted for the mirror 15.

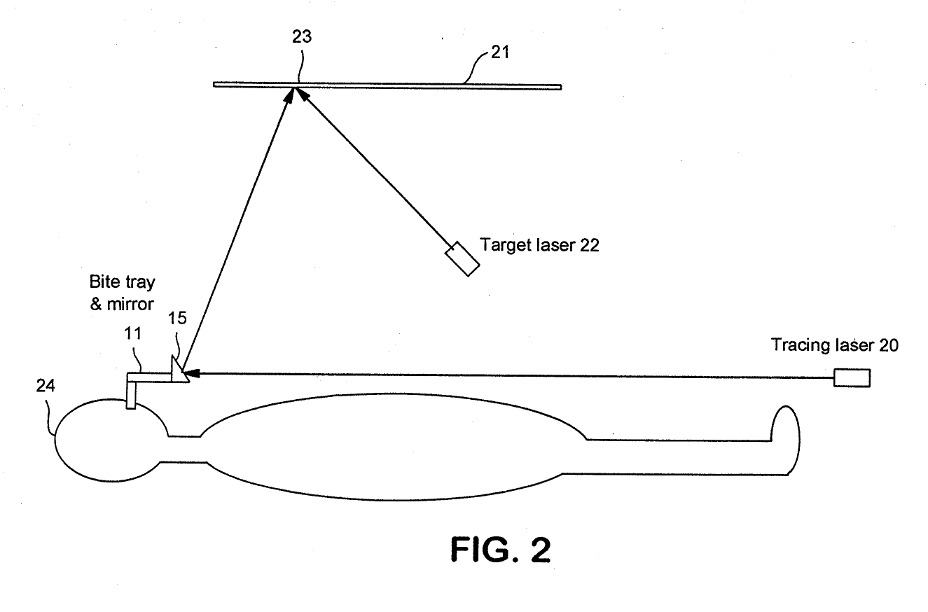

One operation of the method of the invention is depicted in FIG. 2. A tracing laser 20 aims to the mirror 15 on the frame 11 and reflects the patient movement with huge magnification on the wall or ceiling 21 that can be seen by the patient. A target laser 22 provides a target 23 on the wall 21 within which the tracing laser is aimed by the patient by moving his/her head 24. The patient then keeps the tracing laser within the target by constantly repositioning himself/herself throughout the treatment.

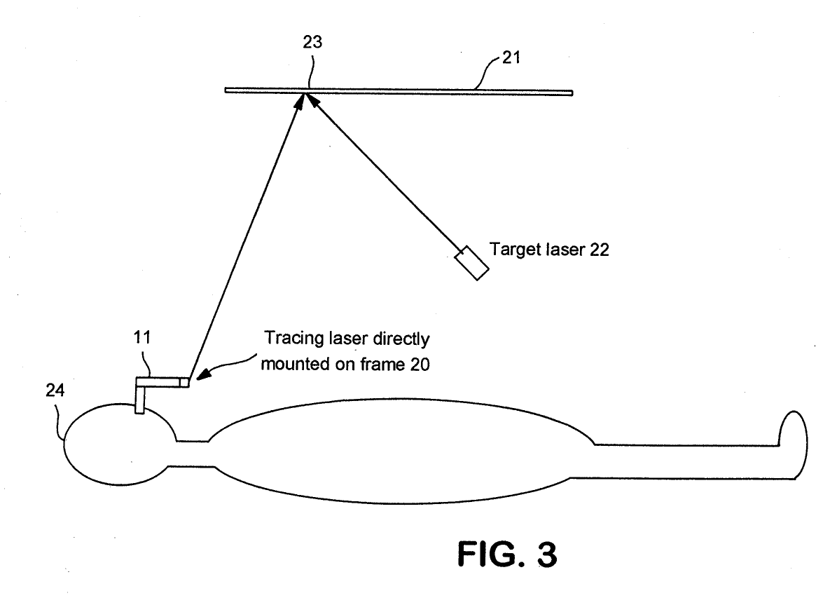

In a second embodiment of the method of the invention, depicted in FIG. 3, the tracing laser 20 is mounted directly on the frame 11. Again, by constantly moving the head 24, the patient maintains the proper position throughout the procedure by aligning the tracing laser with the target 23.

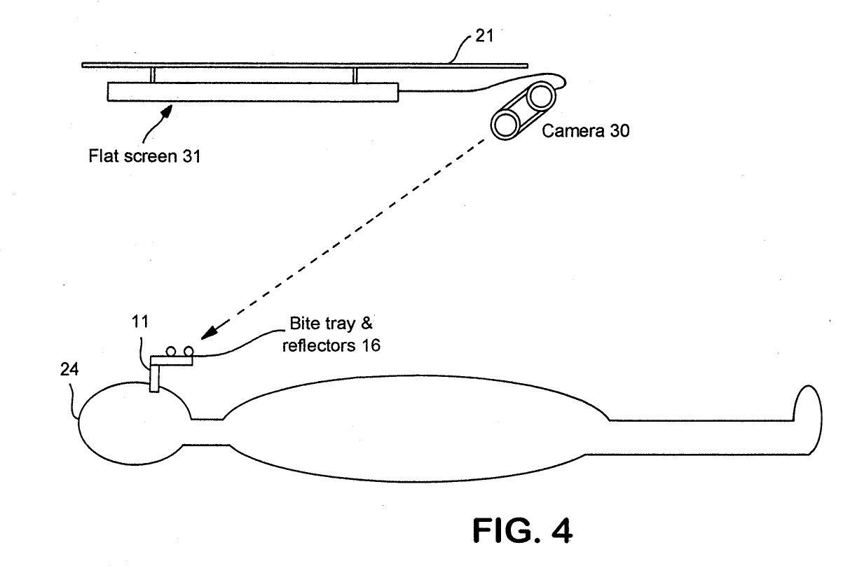

A third embodiment of the method of the invention is depicted in FIG. 4. The frame 11, to which IR reflectors 16 are affixed is held in the patient's mouth. An IR camera 30 provides visual information about the patient's movement that is shown on screen 31. The display of the visual information is the way that the patient can feedback to be moved to the proper position by himself/herself.

The tattoo-free wings 14 enables a valuable function. In a typical CT procedure, a reference point of the patient is determined by three lasers (one sagital and two lateral lasers). Three small radio opaque metal balls are attached on the patient's skin to provide radiographic information of the reference point on the CT image set. Then, three tattoos are made where the radio opaque metal balls are attached to reproduce the setup used for CT scan. The treatment target, however, is generally located somewhere on the patient other than the reference point of CT scan. Thus, it is necessary to shift the patient position with respect to the treatment room coordinates to match the patient's isocenter with that of machine. This is accomplished by setting up the patient based on tattoos made during CT and moving the treatment table the amount required. Then, lasers are lined up with the treatment isocenter and new tattoos are made to reproduce the treatment setup for later treatments. Therefore, most patients have two sets of three tattoos on their skin. This can create problems of confusion from time to time, especially when radiation therapists are changed in the middle of the treatment period that is usually longer than 4 weeks. Precautions must be taken to avoid the treatment of wrong sites.

This is more important for brain, head and neck treatments wherein there are many critical organs close to the tumor. Utilizing the device of the invention, radio opaque metal balls (not shown) are placed on tattoo-free-wings, and the central area of the reflector-frame instead of on the patient's skin. Therefore, patients are not required to have tattoos during CT scans, thereby resolving the problem of confusion between treatment tattoos and CT tattoos.

Intensity modulation radiation therapy (IMRT) is one of the cutting-edge technologies in radiation therapy. The main goal of IMRT is to maximize the avoidance of radiation doses to critical organs and normal tissue surrounding the target while delivering a therapeutic dose to the target volume. To achieve this goal, it inherently requires a very stiff dose gradient between target and critical organs closely located to the target. Both treatment planning and beam delivery require high degrees of precision. Thus, the level of success of IMRT highly depends on how small the overall uncertainties are, how accurately known they are, and how adequately they are incorporated in the planning. These are even more important in the brain head, and neck treatments because there are many critical organs near the target.

The invention is highly valuable in that:
a) It can efficiently improve the accuracy in patient immobilization for the brain, head and neck radiation therapy treatments.
b) It eliminates the possibility of confusion between two sets of reference points, one for CT scan and the other for treatment, when patients are set up for treatment.

What is claimed is:

1. A device for positioning a patient for treatment comprising:
a) a locator attachable to a patient and comprising a registration portion for registration with a portion of a patient's body, and a
b) feedback device that continuously provides patient position feedback to said patient throughout said treatment, said feedback device comprising at least one of a mirror capable of reflecting laser light or a tracing laser generator; wherein said device allows for patient controlled re-positioning at any time during said treatment.

2. The device of claim 1, said locator comprising a bite plate having a first portion fitted with a replaceable dental impression material for engagement with a said patient's teeth and a second portion connected with said feedback device.

3. The device of claim 2 wherein said bite-plate is attached to a frame.

4. The device of claim 3, said frame further comprising tattoo-free wings.

5. The device of claim 1 wherein said means for transmitting information comprise a plurality of spaced apart infrared-reflective markers.

6. An interactive positioning process comprising:
using the device of claim 1 for positioning a patient, the process further comprising:
attaching said locator to said patient, registering said registration portion with a portion of said patient's body, transmitting information concerning the position of said portion of said patient's body using said mirror capable of reflecting laser light or said laser generator; and
providing continuous position feedback to said patient in real-time.

7. The interactive positioning process of claim 6, said locator further comprising: a bite plate having a first portion fitted with a replaceable dental impression material for engagement with the patient's teeth.

8. The interactive positioning process of claim 7, wherein-said patient position is provided relative to a target.

9. The device of claim 1, wherein said device is not used with a head immobilization system.

10. The device of claim 1, wherein said feedback device provides patient position feedback to said patient in real-time.

11. The device of claim 1 wherein said locator is a bite plate having a first portion fitted with a replaceable dental impression material for engagement with a said patient's teeth and a second portion rigidly connected with said feedback device, wherein said locator permits repositioning of said patient during treatment.

12. The device of claim 1 wherein said locator is a bite plate having a first portion fitted with a replaceable dental impression material for engagement with a patient's teeth and a second portion rigidly connected with said feedback device, wherein said feedback device provides real time information concerning said patient's movement during treatment and permits said patient to reposition oneself throughout treatment.

13. A system for positioning a patient in a fixed location comprising the device of claim 1, wherein said feedback device and said locator permits constant repositioning of said patient during said treatment and wherein said treatment is a surgical or medical procedure.

14. The device of claim 3, wherein said mirror is attached to said frame.

15. The device of claim 4, further comprising infra-red reflective markers, wherein said infra-red reflective markers are attached to said frame or said wings.

16. The device of claim 1, further comprising a target, wherein said patient position feedback is provided relative to said target.

17. The device of claim 16, wherein said target is provided by a target laser generator.

18. An interactive positioning process comprising:
using the device of claim 16 for positioning a patient, the process further comprising:
attaching said locator to said patient, said locator further comprising: a bite plate having a first portion fitted with a replaceable dental impression material for engagement with the patient's teeth;
transmitting information concerning the position of said portion of said patient's body using said mirror capable of reflecting laser light or said laser generator; and
providing continuous position feedback to said patient in real-time, wherein said position is relative to said target.

19. The process of claim 18, wherein said target is provided by a target laser generator.

20. The device of claim 1, wherein said treatment is administration of therapy.
